SEQUENCE LISTING (28 sequences)

SEQ ID NO: 1 - DNA, 601 bp, Homo sapiens
tacagtgctataatcacctcctcagatttcttagaccaatgattgtctttaaggattgtatcggcaggactaatttcatattaattgtgttccattatagctagcacgaaggctccatcccatatcttgattttaggaatagacagtgaggaatgccagttaataatcctgtaatgttcaataccttcaccttatatattatgtgtgtatgttttaattgcagtttgaatgatattgttgctgtgggacctgagcacttttatggcacaaatgatcactattttcttgacccctacttacratcctgggagatgtatttgggtttagcgtggtcgtatgttgtctactatagtccaagtgaagttcgagtggtggcagaaggatttgattttgctaatggaatcaacatttcacccgatggcaagtatgtgaactctctgaaatgtagtggatttactcagattctcaggagatatcttggaatatattctttttttaagtaatatgatacattttaacatgttaccctagtgagataaaattaggaaaaatgtaaaatgtcttaatttttcagggggtgaaatttatcaaatcaaaaatt

SEQ ID NO: 2 - DNA, 18 bp, Artificial Sequence
  probe
tgacccctacttacgatc

SEQ ID NO: 3 - DNA, 28 bp, Artificial Sequence
  probe
actattttcttgacccctacttacgatc

SEQ ID NO: 4 - DNA, 38 bp, Artificial Sequence
  probe
acaaatgatcactattttcttgacccctacttacgatc

SEQ ID NO: 5 - DNA, 48 bp, Artificial Sequence
  probe
cttttatggcacaaatgatcactattttcttgacccctacttacgatc

SEQ ID NO: 6 - DNA, 53 bp, Artificial Sequence
  probe
gagcacttttatggcacaaatgatcactattttcttgacccctacttacgatc

SEQ ID NO: 7 - DNA, 54 bp, Artificial Sequence
  probe
tgagcacttttatggcacaaatgatcactattttcttgacccctacttacgatc

SEQ ID NO: 8 - DNA, 10 bp, Artificial Sequence
  probe
acttacgatc

SEQ ID NO: 9 - DNA, 12 bp, Artificial Sequence
  probe
cccctacttaca

SEQ ID NO: 10 - DNA, 18 bp, Artificial Sequence
  probe
cccctacttacaatcctg

SEQ ID NO: 11 - DNA, 28 bp, Artificial Sequence
  probe
ccctacttacaatcctgggagatgtatt

SEQ ID NO: 12 - DNA, 38 bp, Artificial Sequence
  probe
ccctacttacaatcctgggagatgtatttgggtttagc

SEQ ID NO: 13 - DNA, 48 bp, Artificial Sequence
  probe
ccctacttacaatcctgggagatgtatttgggtttagcgtggtcgtat

SEQ ID NO: 14 - DNA, 54 bp, Artificial Sequence
  probe
ccctacttacaatcctgggagatgtatttgggtttagcgtggtcgtatgttgtc

SEQ ID NO: 15 - DNA, 55 bp, Artificial Sequence
  probe
ccctacttacaatcctgggagatgtatttgggtttagcgtggtcgtatgttgtct

SEQ ID NO: 16 - DNA, 21 bp, Artificial Sequence
  primer
gttgctgtgggacctgagcac

SEQ ID NO: 17 - DNA, 25 bp, Artificial Sequence
  primer
tctgccaccactcgaacttcacttg

SEQ ID NO: 18 - DNA, 25 bp, Artificial Sequence
  oligonucleotide
cccaggattgtaagtaggggtcaag

SEQ ID NO: 19 - DNA, 25 bp, Artificial Sequence
  oligonucleotide
cccaggatcgtaagtaggggtcaag

SEQ ID NO: 20 - DNA, 18 bp, Artificial Sequence
  probe
cttgacccctacttacga

SEQ ID NO: 21 - DNA, 399 bp, Homo sapiens
taatcagagaatactacacatgtacaataaaaatttccccatcaagatatacaatatattttatttatatttatagttttaaattacaaccagagcttggcatattgtatctatacctttattaaatgcttttaatttaataaattattgttttctcttagatatgcaataattttcccactatcattgattatttcccrggaacccataacaaattacttaaaaaccttgcttttatggaaagtgatattttggagaaagtaaaagacaccaagaatcgatggacatcaacaaccctcgggactttattgattgcttcctgatcaaaatggagaaggtaaaatgttaacaaaagcttagttatgtgactgcttgcgtatttgtgattcattgactagt

SEQ ID NO: 22 - DNA, 40 bp, Artificial Sequence
  primer
taaattattgttttctcttagatatgcaataattttccca

SEQ ID NO: 23 - DNA, 23 bp, Artificial Sequence
  primer
cccgagggttgttgatgtccatc

SEQ ID NO: 24 - DNA, 17 bp, Artificial Sequence
  probe
tcccaggaacccataac

SEQ ID NO: 25 - DNA, 600 bp, Homo sapiens
atgcatgccaaactcttttttgcttttaagggaattcataggtaagatattacttaaaatttctaaactattattatctgttaacaaatatgaagtgttttatatctaatgtttactcatattttaaaattgtttccaatcatttagcttcaccctgtgatcccactttcatcctgggctgtgctccctgcaatgtgatctgctccattattttccagaaacgtttcgattataaagatcagcaatttcttaacttgatggaaaaattgaatgaaaacatcaggattgtaagcaccccctgratccaggtaaggccaagttttttgcttcctgagaaaccacttacagtctttttttctgggaaatccaaaattctatattgaccaagccctgaagtacatttttgaatactacagtcttgcctagacagccatggggtgaatatctggaaaagatggcaaagttctttattttatgcacaggaaatgaatatcccaatatagatcaggcttctaagcccattagctccctgatcagtgttttttccactaaactccaaagccctgtttctataaagtactttggtgacagccccaaagc

SEQ ID NO: 26 - DNA, 35 bp, Artificial Sequence
  primer
tgatggaaaaattgaatgaaaacatcaggattgta

SEQ ID NO: 27 - DNA, 30 bp, Artificial Sequence
  primer
tcaaaaatgtacttcagggcttggtcaata

SEQ ID NO: 28 - DNA, 17 bp, Artificial Sequence
  probe
ccctgaatccaggtaag

(19) United States
(12) Patent Application Publication
(10) Pub. No.: US 2013/0177908 A1
(43) Pub. Date: Jul. 11, 2013

(54) Probes for Detecting Paraoxonase 1 Gene Polymorphism (Q192R) and Methods of Use Thereof

(71) Applicants: Kaoru Kurose, Kyoto (JP); Mariko Komori, Kyoto (JP)

(72) Inventors: Kaoru Kurose, Kyoto (JP); Mariko Komori, Kyoto (JP)

(73) Assignee: ARKRAY, Inc., Kyoto (JP)

(21) Appl. No.: 13/738,527

(22) Filed: Jan. 10, 2013

(30) Foreign Application Priority Data
Jan. 10, 2012 (JP) 2012-002597

(51) Int. Cl. C12Q 1/68

(52) U.S. Cl. CPC C12Q 1/6883
USPC 435/6.11; 536/24.31

(57) ABSTRACT

The present disclosure provides probes for detecting a polymorphism in the PON1 gene.

### SEQUENCE LISTING SUBMISSION VIA EFS-WEB

A computer readable text file, entitled “SequenceListing.txt,” created on or about Jan. 10, 2013 with a file size of about 8 kb contains the sequence listing for this application and is hereby incorporated by reference in its entirety.

### TECHNICAL FIELD

The present disclosure relates to a method for detecting a paraoxonase 1 (PON1) gene polymorphism (Q192R), a nucleic acid probe and a kit therefor.

### BACKGROUND ART

The PON (paraoxanase) gene cluster is present in the q21.3-q22.1 region on chromosome 7 and encodes three paraoxanase isoforms PON1, PON2 and PON3 having similar structures (The 21st International Symposium on ALS/MND, “Paraoxonase Gene Mutations in Amyotrophic Lateral Sclerosis”, 2010, Enlightenment Committee on ALS Disease, internet <als.gr.jp/staff/sympo/21st_sympo/sympo21_{—}11.html>). Although the three enzymes commonly share functions to protect against hyperoxidation of membrane lipids, each of them also has a specific function. PON1 detoxifies organophosphates and PON2 protects cells against endoplasmic reticulum stress. However, the physiological function of PON3 has not been sufficiently elucidated yet (The 21st International Symposium on ALS/MND, “Paraoxonase Gene Mutations in Amyotrophic Lateral Sclerosis”, 2010, Enlightenment Committee on ALS Disease, internet <als.gr.jp/staff/sympo/21st_sympo/sympo21_{—}11.html>).

The PON1 protein in blood is bound to apoA-1 of HDL (high density lipoprotein), and the protein is considered to inhibit the oxidation of LDL (low density lipoprotein) and suppress the progress of arteriosclerosis (J Clin Invest 96(6): 3005-8, 1995). Further, involvement of PON1 (Q192R) in myocardial infarction and coronary spasm has been suggested (J Clin Invest 96(6): 3005-8, 1995).

Further, PON1 was identified as the most important enzyme in activation of an antiplatelet drug Plavix (generic name: Clopidogrel), and its gene polymorphism Q192R has been revealed to be a determinant of the conversion efficiency into the active form (Nature Medicine 17, 110-116 (2011)). Since it is an antithrombotic drug used for stent therapy, Clopidogrel is commonly used. In Nature Medicine 17, 110-116 (2011), clinical significance of the PON1 Q192R gene polymorphism was investigated in patients with coronary artery disease undergoing stent placement and receiving administration of Clopidogrel. The results suggested that PON1 QQ192 homozygous patients show, when compared to RR192 homozygous patients, higher risk of occurrence of stent thrombosis, lower PON1 activity and Clopidogrel active metabolite in serum, and insufficient inhibition of the platelet activity. Thus, it has been reported that the pharmacological effect of Clopidogrel can be hardly obtained in PON1 QQ192 homozygous patients.

In Nature Medicine 17, 110-116 (2011), the presence/absence of the PON1 (Q192R) mutation was detected using sequence analysis. Extraction of genomic DNA from whole blood for investigation of the pharmacological effect of Clopidogrel is very laborious and costly at actual clinical sites.

JP 2002-119291 A describes a method wherein a nucleic acid probe labeled with a fluorescent dye is allowed to hybridize with a target nucleic acid and the amount of decrease in the luminescence from the fluorescent dye is measured. However, in cases where a nucleic acid probe labeled with a fluorescent dye is allowed to hybridize with a target nucleic acid and the amount of decrease in the luminescence from the fluorescent dye is measured, the sequence of the probe cannot be arbitrary and an appropriate sequence must be found for each mutation.

In Pharmacogenetics 4 1994 pp. 285-299, it is reported that the presence of the homozygous CYP2C19*2 mutation causes splicing abnormality, and this leads to an abnormal structure of the protein, resulting in decreased metabolic efficiencies of various drugs and increased risks of side effects and the like. In Molecular Pharmacology 46 1994 pp. 594-598, it is reported that the presence of the homozygous CYP2C19*3 mutation results in mutation to a stop codon and hence in production of a protein having an abnormal structure by translation, which then leads to decreased metabolic efficiencies of various drugs and increased risks of side effects and the like. Thus, since not only PON1 (Q192R) but also CYP2C19*2 and CYP2C19*3 are involved in the pharmacological effect of Clopidogrel, it is likely that a technique that enables the simultaneous measurement of the presence/absence of PON1 (Q192R) as well as CYP2C19*2 and CYP2C19*3 will be clinically more demanded.

In Pharmacogenetics 4 1994 pp. 285-299, the presence/absence of the CYP2C19*2 mutation was detected using PCR-RFLP. In Molecular Pharmacology 46 1994 pp. 594-598, the presence/absence of the CYP2C19*3 mutation was detected using PCR-RFLP. In JP 2005-58114 A and WO2008/066162, methods for detecting the CYP2C19*2 mutation and nucleic acid probes therefor are described. In JP 2005-110502 A, a method for detecting the CYP2C19*3 mutation and nucleic acid probes therefor are described.

However, none of the above-described prior art documents discloses a simple method for measuring the presence/absence of the PON1 (Q192R) mutation involved in the pharmacological effect of Clopidogrel and a probe to be used therefor, or a method for simultaneously measuring the presence/absence of the PON1 (Q192R) as well as CYP2C19*2 and CYP2C19*3 mutations involved in the pharmacological effect of Clopidogrel.

### SUMMARY

The present disclosure aims to provide probes effective for detection of a PON1 gene polymorphism (Q192R), a method for detecting the PON1 gene polymorphism (Q192R). The disclosure also relates to a method for simultaneously detecting the three mutations PON1 (Q192R), CYP2C19*2 and CYP2C19*3, and a kit therefor.

As described herein, by designing a probe based on a specific region containing the PON1 gene polymorphism (Q192R) and using the probe for detecting a signal due to formation of a hybrid of the probes with target nucleic acids or due to dissociation of the probes from target nucleic acids, the mutation may be detected.

That is, some embodiments of the present disclosure are as follows:

(1) A probe for detecting a polymorphism in the PON1 gene, comprising at least one fluorescently labeled oligonucleotide selected from P1, P1′, P2 and P2′ below:

(P1) an oligonucleotide comprising a nucleotide sequence of 6 to 53 consecutive nucleotides containing nucleotides 299 to 304 in SEQ ID NO: 1 or a homologous sequence thereof, wherein the nucleotide corresponding to the nucleotide at position 304 is a cytosine and is labeled with a fluorescent dye;

(P1′) an oligonucleotide comprising a nucleotide sequence of 6 to 53 consecutive nucleotides containing nucleotides 299 to 304 in SEQ ID NO: 1 or a nucleotide sequence which hybridizes with the complementary strand of the nucleotides 299 to 304 in SEQ ID NO: 1 under stringent conditions, wherein the nucleotide corresponding to the nucleotide at position 304 is a cytosine and is labeled with a fluorescent dye;

(P2) an oligonucleotide comprising a nucleotide sequence of 12 to 54 consecutive nucleotides containing nucleotides 290 to 301 in SEQ ID NO: 1 or a homologous sequence thereof, wherein the nucleotide corresponding to the nucleotide at position 290 is a cytosine and is labeled with a fluorescent dye; and

(P2′) an oligonucleotide comprising a nucleotide sequence of 12 to 54 consecutive nucleotides containing nucleotides 290 to 301 in SEQ ID NO:1 or a nucleotide sequence which hybridizes with the complementary strand of the nucleotides 290 to 301 in SEQ ID NO: 1 under stringent conditions, wherein the nucleotide corresponding to the nucleotide at position 290 is a cytosine and is labeled with a fluorescent dye.

(2) The probe according to (1), wherein

said oligonucleotides P1 and P1′ have the nucleotide corresponding to the nucleotide at position 304 labeled with a fluorescent dye at the first, second or third position counted from the 3′-end; and

said oligonucleotides P2 and P2′ have the nucleotide corresponding to the nucleotide at position 290 labeled with a fluorescent dye at the first, second or third position counted from the 5′-end.

(3) The probe according to (1), wherein

said oligonucleotides P1 and P1′ have the nucleotide corresponding to the nucleotide at position 304 labeled with a fluorescent dye at the 3′-end; and

said oligonucleotides P2 and P2′ have the nucleotide corresponding to the nucleotide at position 290 labeled with a fluorescent dye at the 5′-end.

(4) The probe according to (1), wherein said fluorescently labeled oligonucleotide emits fluorescence when the fluorescently labeled oligonucleotide is not hybridized with a target sequence, and the fluorescence intensity decreases or increases when the fluorescently labeled oligonucleotide is hybridized with a target sequence.
(5) The probe according to (4), wherein said fluorescently labeled oligonucleotide emits fluorescence when the fluorescently labeled oligonucleotide is not hybridized with a target sequence, and the fluorescence intensity decreases when the fluorescently labeled oligonucleotide is hybridized with a target sequence.
(6) The probe according to (1), wherein

said oligonucleotides P1 and P1′ have 6 to 28 consecutive nucleotides; and

said oligonucleotides P2 and P2′ have 12 to 28 consecutive nucleotides.

(7) The probe according to (1), wherein

said oligonucleotides P1 and P1′ have 6 to 18 consecutive nucleotides; and

said oligonucleotides P2 and P2′ have 12 to 18 consecutive nucleotides.

(8) The probe according to (1), wherein said probe is a probe for melting curve analysis.
(9) A method for detecting a polymorphism in the PON1 gene using the probe according to (1). The method may be conducted on a sample comprising nucleic acid which may contain said polymorphism and comprises contacting the probe with said sample and detecting the presence or absence of the polymorphism. The invention also extends to use of said probe for detecting said polymorphism.
(10) A method for detecting a polymorphism in the PON1 gene, comprising the Steps (I) to (IV) below:

(I) adding said probe according to (1) to a sample containing DNA and allowing said probe to hybridize with said DNA;

(II) changing the temperature to dissociate the hybridization complex between said DNA and said probe and measuring the fluctuation of the signal due to said dissociation of said hybridization complex;

(III) analyzing said fluctuation of the signal to determine the Tm value; and

(IV) determining based on said Tm value the presence of the polymorphism of interest.

(11) The method according to (10), further comprising amplifying nucleic acid before said Step (I) or at the same time as said Step (I).
(12) The method according to (9), further comprising detecting in the same system (i.e. in the same reaction or same reaction tube) at least one polymorphism selected from a polymorphism of the nucleotide corresponding to the nucleotide at position 200 in SEQ ID NO:21 and a polymorphism of the nucleotide corresponding to the nucleotide at position 302 in SEQ ID NO:25.
(13) The method according to (9), further comprising detecting in the same system a polymorphism of the nucleotide corresponding to the nucleotide at position 301 in SEQ ID NO:1, a polymorphism of the nucleotide corresponding to the nucleotide at position 200 in SEQ ID NO:21 and a polymorphism of the nucleotide corresponding to the nucleotide at position 302 in SEQ ID NO:25.
(14) A method for judging (or predicting) a pharmacological effect of a drug, comprising detecting a polymorphism in the PON1 gene by the method for detecting a polymorphism according to (9) and judging (or predicting) resistance to said drug and/or a pharmacological effect of said drug based on the presence or absence of said polymorphism detected. Said method may be performed in a sample from a subject (e.g. a human) in relation to whom the judgement/prediction is to be made. A sample (as used herein) comprises nucleic acid (e.g. DNA) which may contain said polymorphism.
(15) A reagent kit for detecting a polymorphism in the PON1 gene, comprising the probe according to (1).
(16) The kit according to (15), further comprising a primer for amplifying a nucleotide sequence comprising the region with which said probe hybridizes.
(17) The kit according to (15), further comprising:

a polymorphism detection probe for detecting a polymorphism of the nucleotide corresponding to the nucleotide at position 200 in SEQ ID NO:21; and

a polymorphism detection probe for detecting a polymorphism of the nucleotide corresponding to the nucleotide at position 302 in SEQ ID NO:25. The invention also extends to the use of said kit for detecting said polymorphism(s).

By including the probe of the present disclosure in a gene amplification system such as PCR and only carrying out Tm analysis after gene amplification reaction, typing of the PON1 gene polymorphism (Q192R) may be carried out. Since the probe of the present disclosure has high specificity, it may be used also in cases where whole blood, an oral mucosa suspension or the like is directly tested. By this, the labour and the costs may be reduced in clinical tests such as tests before dosing.

By using the method of the present disclosure, even in cases where PCR is carried out, the necessity of removal of the amplification product may be eliminated. Therefore, there is hardly any risk of contamination. Further, since the method of the present disclosure may be carried out by a simple procedure, it may be easily automated.

By using the method of the present disclosure, it is also possible to simultaneously detect the three mutations PON1 (Q192R), CYP2C19*2 and CYP2C19*3. In cases where Clopidogrel is used in stent therapy, it is clinically important to be able to simultaneously detect the three mutations.

### BRIEF DESCRIPTION OF THE DRAWINGS

FIG. 1 shows diagrams showing examples of (A) a melting curve of a nucleic acid mixture; and (B) a differential melting curve.

FIG. 2 shows melting curves obtained using the probe of Example 1 of the present disclosure for detecting a polymorphism. The amount of change in the fluorescence intensity per unit time is plotted along the ordinate (d the amount of increase in the fluorescence intensity/t) and the temperature (° C.) is plotted along the abscissa. Squares represent the wild-type, diamonds represent the mutant type, and triangles represent the heterozygous type (this also applies to the figures below).

FIG. 3 shows melting curves obtained using the probe of Example 2 for detecting a polymorphism.

FIG. 4 shows melting curves obtained using the probe of Comparative Example 1 for detecting a polymorphism.

FIG. 5 shows melting curves obtained from purified human genomic DNA using the probes of Example 3 for detecting polymorphisms. The left panel shows a melting curve for detecting the PON1 gene polymorphism (Q192R); the middle panel shows a melting curve for detecting the CYP2C19*2 gene polymorphism; and the right panel shows a melting curve for detecting the CYP2C19*3 gene polymorphism (this also applies to the figure below).

FIG. 6 shows melting curves obtained from whole blood using the probes of Example 3.

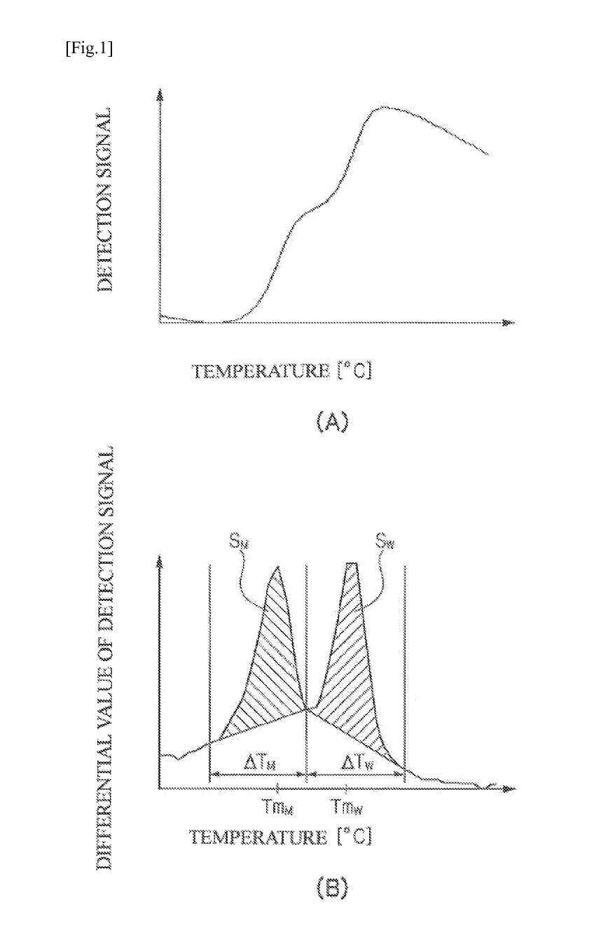

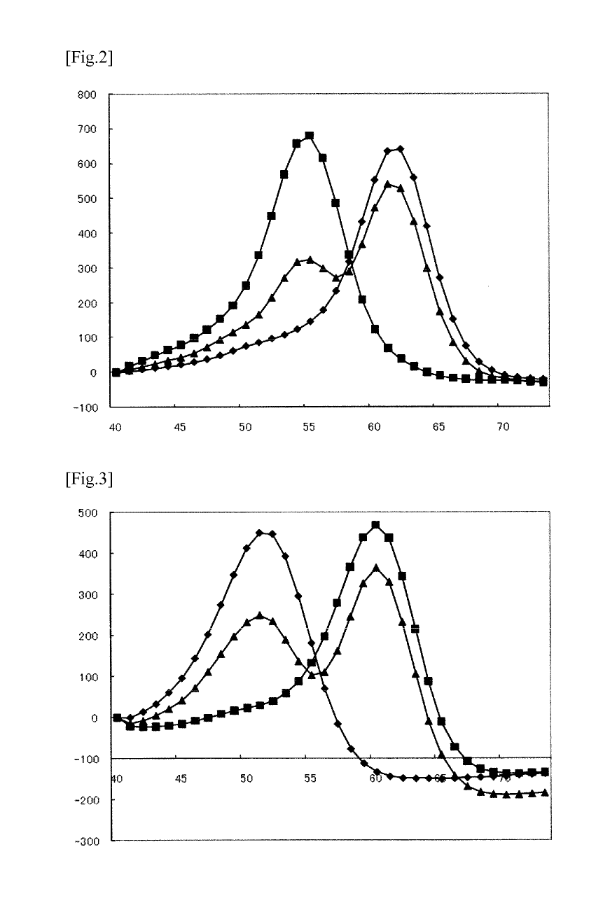

### MODE FOR CARRYING OUT THE INVENTION

### <1> Probes and Detection Method of Present Disclosure

The (P1) probe is a probe for detecting the PON1 gene polymorphism (Q192R) (that is, rs662), and is composed of an oligonucleotide comprising a nucleotide sequence of 6 to 53 consecutive nucleotides containing nucleotides 299 to 304 in SEQ ID NO:1 or a homologous sequence thereof. In some embodiments, the nucleotide corresponding to the nucleotide at position 304 is a cytosine and is labeled with a fluorescent dye. As referred to in this context, the homologous sequence is homologous to SEQ ID NO:1 or the recited portion of SEQ ID NO:1. Thus, said oligonucleotide has 6 to 53 nucleotides derived from said homologous sequence, including nucleotides corresponding to nucleotides 299 to 304 in SEQ ID NO:1. The 6 to 53 nucleotides are all contained, consecutively in SEQ ID NO:1 or its homologous sequence.

The (P1′) probe is a probe for detecting the PON1 gene polymorphism (Q192R) (that is, rs662), and is composed of an oligonucleotide comprising a nucleotide sequence of 6 to 53 consecutive nucleotides containing nucleotides 299 to 304 in SEQ ID NO:1 or a nucleotide sequence which hybridizes with the complementary strand of the nucleotides 299 to 304 in SEQ ID NO: 1 under stringent conditions, wherein the nucleotide corresponding to the nucleotide at position 304 is a cytosine and is labeled. In some embodiments, the nucleotide corresponding to the nucleotide at position 304 is labeled with a fluorescent dye. As referred to in this context, the hybridizing sequence hybridizes with the complementary strand of SEQ ID NO:1 or the recited portion of SEQ ID NO:1. Thus, said oligonucleotide has 6 to 53 nucleotides derived from said hybridizing sequence, including nucleotides corresponding to nucleotides 299 to 304 in SEQ ID NO:1. The 6 to 53 nucleotides are all contained, consecutively in SEQ ID NO:1 or the hybridizing sequence.

The (P1) and (P1′) probes may also be peptide nucleic acid (PNA) having amide-linked N-(2-aminoethyl)glycine as the backbone instead of the deoxyribose backbone of an oligonucleotide, or may be nucleic acid having two cyclic structures wherein the oxygen atom at the 2′-position and the carbon atom at the 4′-position in the sugar of nucleic acid are cross-linked (LNA; Locked Nucleic Acid).

The PON1 gene polymorphism rs662 described herein is located at nucleotide position 301 in SEQ ID NO:1. The rs number represents a registration number for the dbSNP database curated by the National Center for Biotechnology Information (ncbi.nlm.nih.gov/projects/SNP/). The nucleotide at position 301 in SEQ ID NO:1 is represented as r, which means the nucleotide A in the wild-type and the nucleotide G in the mutant type. For example, in the (P1) and (P1′) probe, the nucleotide at position 301 in SEQ ID NO:1 is G, which is the mutant-type nucleotide.

In additional embodiments, the probe for detecting a polymorphism in a paraoxonase 1 (PON1) gene, comprising a labeled oligonucleotide selected from P1, P1′, P2 and P2′ below:

(P1) an oligonucleotide of any length from 6 to 53 nucleotides comprising a sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to nucleotides 299 to 304 in SEQ ID NO: 1, wherein the nucleotide corresponding to the nucleotide at position 304 in SEQ ID NO: 1 is a cytosine and is labeled;

(P1′) an oligonucleotide of any length from 6 to 53 nucleotides which hybridizes with the complementary nucleotide sequence to the nucleotides 299 to 304 in SEQ ID NO: 1 under stringent conditions, wherein the nucleotide complementary to the nucleotide at position 304 in SEQ ID NO: 1 is a cytosine and is labeled;

(P2) an oligonucleotide of any length from 12 to 54 nucleotides comprising a sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical to nucleotides 290 to 301 in SEQ ID NO: 1, wherein the nucleotide corresponding to the nucleotide at position 290 in SEQ ID NO: 1 is a cytosine and is labeled; and

(P2′) an oligonucleotide of any length from 12 to 54 nucleotides which hybridizes with the complementary nucleotide sequence to the nucleotides 290 to 301 in SEQ ID NO: 1 under stringent conditions, wherein the nucleotide complementary to the nucleotide at position 290 is a cytosine and is labeled.

The (P1) and (P1′) probes may be prepared in the same manner as the probe described in JP 2002-119291 A except that the (P1) and (P1′) probes has the above-described specified sequence in the nucleotide sequence shown in SEQ ID NO:1. As shown in Table 1, the length of the (P1) and (P1′) probe is, for example, 6 to 53 consecutive nucleotides, 10 to 30 consecutive nucleotides or 13 to 25 consecutive nucleotides. In the present disclosure, for example, the mutation of the nucleotide at position 301 in SEQ ID NO:1 may be more sensitively detected in cases where the difference between the Tm value observed when the (P1) oligonucleotide is hybridized with DNA having the complementary nucleotide sequence and the Tm value observed when the (P1) oligonucleotide is hybridized with DNA having the complementary nucleotide sequence except that only one nucleotide at position 301 in SEQ ID NO:1 is not complementary is not less than 2.0° C.

Further, as shown in Table 2, in cases where an artificial nucleic acid such as LNA is used, a probe having not less than 6 consecutive nucleotide is enough to obtain Tm values with which mutation of the nucleotide at position 301 in SEQ ID NO:1 may be detected.

TABLE 1

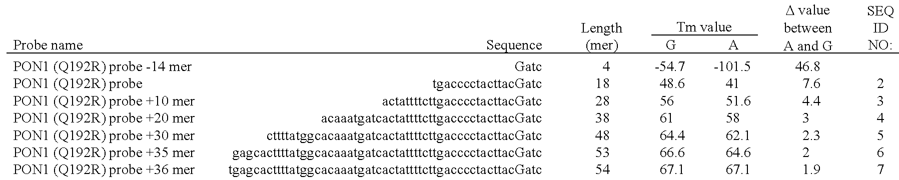

*The nucleotides denoted by the upper case letters indicate the position of the mutation.

The Tm value was calculated with Meltcalc 99 free (meltcalc.com/) under the conditions of: Oligoconc [μM] 0.2, Na eq. [mM] 50.

TABLE 2

| | | | | SEQ |
|---|---|---|---|---|
| | | Length | Tm value | ID |
| | Sequence | (mer) | G | NO: |
| PON1 (Q192R) probe | Gatc | 4 | ? | |
| using LNA | acGatc | 6 | 38 | |
| | ttacGatc | 8 | 57 | |
| | acttacGatc | 10 | 70 | 8 |

*The nucleotides denoted by the upper case letters indicate the position of the mutation.

Table 2 describes the Tm values observed when LNA was used. By using LNA, the Tm value may be calculated even in cases of 6 consecutive nucleotides. The Tm value was calculated using LNA™ Oligo Tools and Design Guidelines (exiqon.com/oligo-tools).

Examples of the nucleotide sequence of the (P1) probe used in the present disclosure include 5′-tgacccctacttacGatc-3′ (SEQ ID NO:2), in which the upper-case nucleotide G corresponds to the nucleotide at position 301.

The (P2) probe is a probe for detecting the PON1 gene polymorphism (Q192R) (that is, rs662), and is composed of an oligonucleotide comprising a nucleotide sequence of 12 to 54 consecutive nucleotides containing nucleotides 290 to 301 in SEQ ID NO:1 or a homologous sequence thereof, wherein the nucleotide corresponding to the nucleotide at position 290 is a cytosine and is labeled. In some embodiments, the nucleotide corresponding to the nucleotide at position 290 is labeled with a fluorescent dye. As referred to in this context, the homologous sequence is homologous to SEQ ID NO:1 or the recited portion of SEQ ID NO:1. Thus, said oligonucleotide has 12 to 54 nucleotides derived from said homologous sequence, including nucleotides corresponding to nucleotides 290 to 301 in SEQ ID NO:1. The 12 to 54 nucleotides are all contained, consecutively in SEQ ID NO:1 or its homologous sequence.

The (P2′) probe is a probe for detecting the PON1 gene polymorphism (Q192R) (that is, rs662), and is composed of an oligonucleotide comprising a nucleotide sequence of 12 to 54 consecutive nucleotides containing nucleotides 290 to 301 in SEQ ID NO:1 or a nucleotide sequence which hybridizes with the complementary strand of the nucleotides 290 to 301 in SEQ ID NO: 1 under stringent conditions, wherein the nucleotide corresponding to the nucleotide at position 290 is a cytosine and is labeled. In some embodiments, the nucleotide corresponding to the nucleotide at position 290 is labeled with a fluorescent dye. As referred to in this context, the hybridizing sequence hybridizes with the complementary strand of SEQ ID NO:1 or the recited portion of SEQ ID NO:1. Thus, said oligonucleotide has 12 to 54 nucleotides derived from said hybridizing sequence, including nucleotides corresponding to nucleotides 290 to 301 in SEQ ID NO:1. The 12 to 54 nucleotides are all contained, consecutively in SEQ ID NO:1 or the hybridizing sequence.

The (P2) and (P2′) probes may also be peptide nucleic acid (PNA) having amide-linked N-(2-aminoethyl)glycine as the backbone instead of the deoxyribose backbone of an oligonucleotide, or may be nucleic acid having two cyclic structures wherein the oxygen atom at the 2′-position and the carbon atom at the 4′-position in the sugar of nucleic acid are cross-linked (LNA; Locked Nucleic Acid).

For example, in the (P2) and (P2′) probes of the present disclosure, the nucleotide at position 301 in SEQ ID NO:1 is A, which is the wild-type nucleotide.

The (P2) and (P2′) probes may be prepared in the same manner as the quenching probe described in JP 2002-119291 A except that the (P2) and (P2′) probes of the present disclosure has the above-described specified sequence in the nucleotide sequence shown in SEQ ID NO:1. As shown in Table 3, the length of the probe of the present disclosure is, for example, 12 to 54 consecutive nucleotides, 12 to 30 consecutive nucleotides or 13 to 25 consecutive nucleotides.

TABLE 3

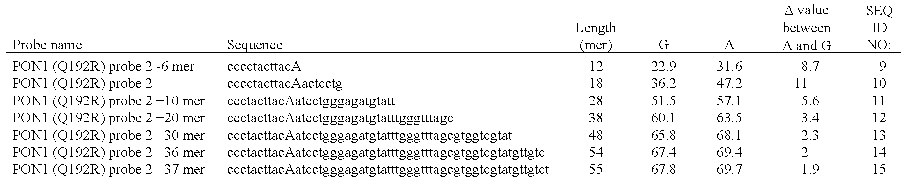

*The nucleotides denoted by the upper case letters indicate the position of the mutation.

The Tm value was calculated with Meltcalc 99 free (meltcalc.com/) under the conditions of: Oligoconc [μM] 0.2, Na eq. [mM] 50.

In the present disclosure, for example, the mutation of the nucleotide at position 301 in SEQ ID NO:1 may be more sensitively detected in cases where the difference between the Tm value observed when the (P2) oligonucleotide is hybridized with DNA having the complementary nucleotide sequence and the Tm value observed when the (P2) oligonucleotide is hybridized with DNA having the complementary nucleotide sequence except that only one nucleotide at position 301 in SEQ ID NO:1 is not complementary is not less than 2.0° C.

Examples of the nucleotide sequence of the (P2) probe used in the present disclosure include 5′-cccctacttacAatcctg-3′ (SEQ ID NO:10), in which the upper-case nucleotide A corresponds to the nucleotide at position 301.

The fluorescent dye is not restricted, and examples of the fluorescent dye include fluorescein, phosphor, rhodamine and polymethine dye derivatives. Examples of commercially available fluorescent dyes include BODIPY FL (manufactured by Molecular Probes), FluorePrime (manufactured by Amersham Pharmacia), Fluoredite (trade name; manufactured by Millipore), FAM (manufactured by ABI), Cy3 and Cy5 (manufactured by Amersham Pharmacia) and TAMRA (manufactured by Molecular Probes). The conditions for detection using the probe are not restricted, and may be appropriately determined depending on the fluorescent dye used. The detection may be carried out, for example, with a detection wavelength of 445 to 480 nm in the case of Pacific Blue, with a detection wavelength of 585 to 700 nm in the case of TAMRA, and with a detection wavelength of 520 to 555 nm in the case of BODIPY FL. By using such a probe, hybridization and dissociation may be easily confirmed based on fluctuation of the signal. The fluorescent dye may be bound to the oligonucleotide by a conventional method such as the method described in JP 2002-119291 A.

The “homologous sequence” in the present specification means a nucleotide sequence having a sequence having a homology (or identity) of, for example, not less than 80%, 81%, 82%, 83%, 84%, 85%, 86%, 87%, 88%, 89%, 90%, 91%, 92%, 93%, 94%, 95%, 96%, 97%, 98% or 99% to a particular nucleotide sequence. The identity may also be 100% in the present application.

The hybridization described herein may be carried out according to a known method or a method corresponding thereto, such as the method described in Molecular Cloning 3rd (J. Sambrook et al., Cold Spring Harbor Lab. Press, 2001). This literature is hereby incorporated in the present specification by reference. Said hybridization is under stringent conditions, for example.

The term “stringent conditions” means conditions under which a specific hybrid is formed while nonspecific hybrids are not formed. Typical examples of stringent conditions include conditions under which hybridization is performed with a potassium concentration of about 25 mM to about 50 mM and a magnesium concentration of about 1.0 mM to about 5.0 mM. Examples of the conditions in the present disclosure include conditions under which hybridization is performed in Tris-HCl (pH 8.6), 25 mM KCl and 1.5 mM MgCl₂, but the conditions are not limited thereto. Other examples of stringent conditions include those described in Molecular Cloning 3rd (J. Sambrook et al., Cold Spring Harbor Lab. Press, 2001). This literature is hereby incorporated in the present specification by reference. Those skilled in the art may easily select such conditions by, for example, controlling the hybridization reaction and/or changing salt concentrations of the hybridization reaction solution.

Examples of the oligonucleotide of the above fluorescently labeled oligonucleotide include oligonucleotides as well as modified oligonucleotides.

Examples of the constituent units of the above oligonucleotide include ribonucleotides, deoxyribonucleotides and artificial nucleic acids. Examples of the artificial nucleic acids include DNA; RNA; LNA (Locked Nucleic Acid), which is an RNA analogue; PNA (Peptide Nucleic Acid), which is a peptide nucleic acid; and BNA (Bridged Nucleic Acid), which is a bridged nucleic acid.

The oligonucleotide may be constituted by either a single type of constituent unit or a plurality of types of constituent units among the above constituent units.

The (P1) and (P1′) probes have a nucleotide sequence of 6 to 53 consecutive nucleotide containing nucleotides 299 to 304 in SEQ ID NO:1, and have homology to SEQ ID NO:1 except that the nucleotide at position 304 is a cytosine. That is, although the (P1) and (P1′) probes are homologous to the nucleotide sequence of 6 to 53 consecutive nucleotides containing nucleotides 299 to 304, the probe does not need to be completely identical thereto.

The (P2) and (P2′) probes has a nucleotide sequence of 12 to 54 consecutive nucleotides containing nucleotides 290 to 301 in SEQ ID NO: 1, and have homology to SEQ ID NO:1 except that the nucleotide at position 290 is a cytosine. That is, although the (P2) and (P2′) probes are homologous to the nucleotide sequence of 12 to 54 consecutive nucleotides containing nucleotides 290 to 301 in SEQ ID NO:1, the probe does not need to be completely identical thereto.

For example, the fluorescently labeled oligonucleotide is a fluorescently labeled oligonucleotide whose fluorescence intensity decreases (or the fluorescence is quenched) or increases when the oligonucleotide is hybridized with a complementary sequence, relative to the fluorescence intensity observed when the oligonucleotide is not hybridized with the complementary sequence. For example, the fluorescence intensity of the fluorescently labeled oligonucleotide decreases when the oligonucleotide is hybridized with a complementary sequence, relative to the fluorescence intensity observed when the oligonucleotide is not hybridized with the complementary sequence. A probe that utilizes such a fluorescence quenching phenomenon is generally called a guanine quenching probe, and is known as a QPROBE. For example, the fluorescently labeled oligonucleotide is an oligonucleotide which is designed such that the 3′-end or 5′-end of the oligonucleotide is C, which terminal C is labeled with a fluorescent dye such that the emission decreases when the terminal C comes close to G.

Further, the probe of the present disclosure is, for example, labeled with a fluorescent dye at its 5′- or 3′-end.

In the present specification, when the term “first, second or third position counted from the 5′-end” is mentioned, the 5′-end is regarded as the first position. Further, when the term “first, second or third position counted from the 3′-end” is mentioned, the 3′-end is regarded as the first position.

In the detection method of the present disclosure, nucleic acid comprising the PON1 gene polymorphism (Q192R), and/or the CYP2C19*2 and/or CYP2C19*3 polymorphism(s) is analyzed using a nucleic acid probe labeled with a fluorescent dye(s) by measuring fluorescence from the fluorescent dye(s), to perform melting curve analysis, followed by detecting the polymorphism(s) based on the result of the melting curve analysis, which nucleic acid probe is the probe disclosed herein.

The detection method of the present disclosure uses the probe of the present disclosure and comprises the following steps:

(I) adding the probe of the present disclosure to a sample containing DNA and allowing the probe to hybridize with the DNA;

(II) changing the temperature to dissociate the hybridization complex between the DNA and the probe and measuring the fluctuation of the signal due to the dissociation of the hybridization complex;

(III) analyzing the fluctuation of the signal to determine the Tm value; and

(IV) determining based on the Tm value the presence or absence of the polymorphism of interest.

The evaluation of the Tm value in (III) includes not only evaluation of the dissociation temperature of the hybridization complex but also evaluation of the differential value of the fluorescence signal which fluctuates upon melting of the hybridization complex depending on the temperature.

Examples of the method of nucleic acid amplification include methods using a polymerase, such as PCR, ICAN and LAMP. When the amplification is carried out by a method using a polymerase, the amplification may be carried out in the presence of the probe of the present disclosure. By this, the detection may be carried out just by analyzing the Tm value of the probe after the amplification of the nucleic acid, so that the amplification product does not need to be handled after the reaction. Therefore, there is no risk of contamination of the amplification product. Further, since the detection may be carried out with the same apparatus as the one necessary for the amplification, it is not necessary to transfer the container. Therefore, automation may also be easily done.

As the DNA polymerase to be used for the PCR method, a conventional DNA polymerase may be used without any limitation. Examples of the DNA polymerase include GeneTaq (manufactured by Nippon Gene Co., Ltd.), PrimeSTAR Max DNA Polymerase (manufactured by Takara Bio Inc.) and Taq Polymerase.

The amount of the polymerase to be used is not restricted as long as the polymerase is used at a concentration which is normally employed. For example, in cases where Taq polymerase is used, the concentration of the polymerase may be 0.01 U to 100 Upper 50 μl of the reaction solution. This has an advantage, for example, in that a sufficient amount of amplified product may be obtained.

The DNA in the sample may be either single-stranded DNA or double-stranded DNA. In cases where the DNA is double-stranded DNA, for example, the step of dissociating the double-stranded DNA in the sample by heating may be included before the hybridization step. By dissociating the double-stranded DNA into single-stranded DNA, hybridization with the fluorescently labeled oligonucleotide is possible.

The ratio (molar ratio) of the probe of the present disclosure to be added with respect to the DNA in the sample is not restricted, and the ratio is, for example, not more than 1 or not more than 0.3 with respect to the DNA in the sample to ensure a sufficient detection signal. In this case, for example, the DNA in the sample may be either: the sum of the target DNA wherein the polymorphism to be detected is present and non-target DNA wherein the polymorphism to be detected is absent; or the sum of the amplification product containing the target sequence wherein the polymorphism to be detected is present and the amplification product containing the non-target sequence wherein the polymorphism is absent. Although the ratio of the DNA to be detected relative to the DNA in the sample is usually not known, the ratio (molar ratio) of the probe to be added with respect to the DNA to be detected (the amplification product containing the sequence to be detected) is, for example, not more than 100, not more than 50, or not more than 30, as a result. The lower limit of the ratio is not restricted, and the ratio is, for example, not less than 0.001, not less than 0.01, or not less than 0.2. The ratio of the probe of the present disclosure to be added with respect to the DNA may be, for example, either the molar ratio with respect to the double-stranded DNA or the molar ratio with respect to the single-stranded DNA.

The Tm value will now be described. Heating a solution containing double-stranded DNA causes an increase in absorbance at 260 nm. This is caused because the hydrogen bonds between the strands of the double-stranded DNA are broken by the heat and the double-stranded DNA is dissociated into single-stranded DNA (melting of DNA). When the double-stranded DNA is completely dissociated into single-stranded DNA, the absorbance is about 1.5-fold higher than the absorbance at the beginning of heating (absorbance for only the double-stranded DNA), and completion of the melting may be judged by this. Based on this phenomenon, the melting temperature Tm is generally defined as the temperature at which the increase in absorbance reached 50% of the total increase in absorbance.

Measurement of the signal fluctuation due to the temperature change for determination of the Tm value may be carried out also by measuring absorbance at 260 nm based on the above-mentioned principle, but the measurement may be carried out based on a signal from a label added to the probe of the present disclosure, which signal fluctuates depending on the state of hybridization between the DNA and the probe. Therefore, as the probe of the present disclosure, the above-mentioned labeled probe may be used. Examples of the labeled probe include a fluorescently labeled oligonucleotide probe which emits fluorescence when it is not hybridized with the target sequence, whose fluorescence intensity decreases (or the fluorescence is quenched) when the probe is hybridized with the target sequence, and a fluorescently labeled oligonucleotide probe which emits fluorescence when it is not hybridized with the target sequence, whose fluorescence intensity increases when the probe is hybridized with the target sequence. In the case of the former probe, the probe shows no signal or shows a weak signal when it forms a hybrid (double-stranded DNA) with the sequence to be detected, while the probe shows a signal or the signal increases when the probe is released by heating. In the case of the latter probe, the probe shows a signal when forming a hybrid (double-stranded DNA) with the sequence to be detected, while the signal decreases (disappears) when the probe is released by heating. Therefore, by detecting the change in the signal from the label under conditions specific to the signal (by investigating absorbance or the like), the progress of melting and the Tm value may be determined in a similar manner to the measurement of absorbance at 260 nm. For example, the labeling substance in the labeled probe is as mentioned above, and the probe may be labeled with a fluorescent dye.

The method of detecting a polymorphism will now be described by way of specific examples of the method by detection of changes in the signal from a fluorescent dye. It should be noted that the method of detecting a polymorphism may be characterized by the use per se of the probe for detection of a polymorphism, and other steps and conditions are not restricted.

The range of the temperature at which fluctuation of the fluorescence intensity is measured is not restricted, and the starting temperature may be, for example, room temperature to 85° C., or 25° C. to 70° C., and the end temperature may be, for example, 40° C. to 105° C. The heating rate is not restricted, and may be, for example, 0.1° C./second to 20° C./second, or 0.3° C./second to 5° C./second.

The fluctuation of the signal is then analyzed to determine the Tm value. More particularly, the differential value at each temperature (−d the fluorescence intensity/dt) is calculated based on the obtained fluorescence intensity, and the temperature at which the value is lowest may be determined as the Tm value. Further, the point at which the amount of increase in the fluorescence intensity per unit time (the amount of increase in the fluorescence intensity/t) is largest may also be determined as the Tm value. In cases where, as the labeled probe, a probe with which the signal intensity increases upon hybridization is used instead of a quenching probe, the amount of decrease in the fluorescence intensity may be measured instead.

In the presently described method, the hybridization complex is heated and fluorescence signal fluctuation (for example, an increase in the fluorescence intensity) due to the increase in the temperature is measured as described above, but, instead of employing this method, signal fluctuation upon hybridization may be measured, for example. That is, fluorescence signal fluctuation caused by formation of the hybridization complex due to the decrease in the temperature of the sample to which the probe was added may be measured.

For example, in cases where a fluorescently labeled oligonucleotide probe whose fluorescence intensity is lower (or whose fluorescence is quenched) when the probe is hybridized with the complementary sequence than when the probe is not hybridized with the complementary sequence (e.g., QProbe) is used, the fluorescence intensity is high upon addition of the probe to the sample since the probe is in the dissociated state, while the fluorescence decreases (or is quenched) upon formation of a hybridization complex due to the decrease in the temperature. Therefore, for example, the temperature of the heated sample may be gradually decreased, to measure the decrease in the fluorescence intensity due to the temperature decrease.

On the other hand, in cases where a labeled probe whose signal increases upon formation of the hybrid (e.g. TaqMan probe manufactured by Roche Molecular Systems, Inc.) is used, the fluorescence intensity is low (or the fluorescence is quenched) upon addition of the probe to the sample since the probe is in the dissociated state, while the fluorescence intensity increases upon formation of a hybridization complex due to the decrease in the temperature. Therefore, for example, the temperature of the sample may be gradually decreased, to measure the increase in the fluorescence intensity due to the temperature decrease.

The nucleic acid to be used as a template for carrying out the nucleic acid amplification is not restricted as long as it contains nucleic acid, and examples of the nucleic acid include those derived from, or those which may be derived from, arbitrary biological origins such as blood; oral mucosal suspensions; somatic cells of nails, hair and the like; germ cells; milk; ascitic fluid; paraffin-embedded tissues; gastric juices; fluids obtained by gastric lavage; peritoneal fluid; amniotic fluid; and cell cultures. The nucleic acid as a template may be used as it is directly after being obtained from any of the above-mentioned origins, or may be pretreated to modify properties of the sample before being used. For example, in cases where whole blood is used as a sample, isolation of genomic DNA from the whole blood may be carried out by a known method. For example, a commercially available genomic DNA isolation kit (trade name, GFX Genomic Blood DNA Purification kit; manufactured by GE Healthcare Bio-Sciences KK) or the like may be employed.

The primer pair used in the PCR is not restricted as long as a region with which the probe of the present invention can be hybridized is amplified. Those skilled in the art can easily design such a primer pair based on the nucleotide sequence of SEQ ID NO:1. The length and the Tm value of each primer are usually 12-mer to 40-mer and 40 to 70° C., or 16-mer to 30-mer and 55 to 60° C., respectively.

The primers in the primer pair do not need to have the same length, but the Tm values of these primers are almost the same (or the difference is not more than 5° C.), for example.

The Tm analysis may be carried out in the same manner as in the conventional method except that fluorescence from the fluorescent dye of the probe of the present disclosure is measured. The fluorescence may be measured using the excitation light having a wavelength dependent on the fluorescent dye while measuring the light having the emission wavelength. The heating rate in the Tm analysis is usually 0.3 to 5° C./second. The composition of the reaction solution used for carrying out the Tm analysis is not restricted as long as the probe may hybridize with nucleic acid having the complementary sequence of the nucleotide sequence of the probe, and usually, the concentration of monovalent cations is 1.5 to 5 mM, and the pH is 7 to 9. Since the reaction solution in an amplification method using a DNA polymerase, such as PCR, usually satisfies these conditions, the reaction solution after the amplification may be used as it is for the Tm analysis.

Detection of the PON1 gene polymorphism (Q192R) and the CYP2C19*2 and CYP2C19*3 gene polymorphisms based on the result of the Tm analysis may be carried out according to a conventional method. The detection in the present disclosure includes detection of the presence/absence of a polymorphism (s).

Since detection of the presence/absence of a polymorphism (s) is possible with the probe and by the method of the present disclosure, judgment (or prediction) of the pharmacological effect of Clopidogrel and resistance to Clopidogrel based on the presence/absence of a polymorphism (s) is also included in the present disclosure. More specifically, in cases where the nucleotide at the PON1 gene polymorphism rs662 is the wild-type A (A/A), it is suggested that Clopidogrel is less likely to be effective and the risk of stent thrombosis is high, while in cases where the mutant type G (G/G) is contained, the risk is suggested to be low. Further, in cases where the CYP2C19*2(G681A) or CYP2C19*3(G636A) mutation is homozygous (that is, CYP2C19*2 is A/A or CYP2C19*3 is A/A), it is suggested that drugs such as Clopidogrel show decreased metabolic efficiencies and the risk of side effects is high.

Thus, by the present disclosure, all of the PON1 gene polymorphism (Q192R) and the CYP2C19*2 and CYP2C19*3 gene polymorphisms may be detected at once.

The polymorphism probes for the CYP2C9*2 and CYP2C9*3 mutation sites which may be used in simultaneous detection (that is, detection in the same system) of the PON1 gene polymorphism are described in, for example, JP 2005-58114 A, WO2008/066162 and JP 2005-110502 A. More specifically, each of the probes is a nucleic acid probe labeled with a fluorescent dye at the 5′-end or 3′-end, and fluorescence from the fluorescent dye decreases upon occurrence of hybridization. The nucleic acid probes have the nucleotide sequences shown in SEQ ID NO:64 and SEQ ID NO:76 in WO2008/066162. The nucleotide sequences shown in SEQ ID NO:64 and SEQ ID NO:76 in WO2008/066162 are the same as the nucleotide sequences shown in SEQ ID NO:24 and SEQ ID NO:28 in the present disclosure.

### <2> Kit of

The kit described herein is a kit to be used for the detection method also described herein. This kit may comprise a nucleic acid probe composed of the above-described oligonucleotide. In some embodiments, the probe is labeled with a fluorescent dye whose fluorescence changes upon hybridization (e.g., quenching probe). The kit of the present disclosure may also be used for judging (or predicting) the pharmacological effect of Clopidogrel and resistance to Clopidogrel.

The detection kit of the present disclosure may further comprise, in addition to the probe, reagents required for nucleic acid amplification in the detection method of the present disclosure, especially a primer(s) for amplification using a DNA polymerase.

More specifically, the primers of the present disclosure are primers for amplifying a region in the nucleotide sequence shown in SEQ ID NO:1 in the PON1 gene, which region comprises a sequence with which the P1, P1′, P2 or P2′ oligonucleotide hybridizes (e.g., SEQ ID NOs:16 and 17). Primers for amplifying a region in the nucleotide sequence shown in SEQ ID NO:21 in the CYP2C19*2 gene, which region comprises a sequence with which the oligonucleotide shown in SEQ ID NO:24 hybridizes (e.g., SEQ ID NOs:22 and 23); and/or primers for amplifying a region in the nucleotide sequence shown in SEQ ID NO:25 in the CYP2C19*3 gene, which region comprises a sequence with which the oligonucleotide shown in SEQ ID NO:28 hybridizes (e.g., SEQ ID NOs:26 and 27); are also included in the present disclosure.

In the detection kit of the present disclosure, the probe, primer and other reagents may be stored separately, or a part of these components may be stored as a mixture(s).

The present disclosure will now be described concretely by way of Examples. However, these Examples are merely examples and the present disclosure is not restricted thereto. It will be apparent to those skilled in the art that various modifications and variations can be made in the present disclosure without departing from the spirit or scope of the inventions. Thus, it is intended that the present disclosure covers the modifications and variations of this invention provided they come within the scope of the appended claims and their equivalents.

In the present disclosure, in terms of the individual sequences of the sample nucleic acid in the sample to be subjected to the detection, and the probes and primers for detecting polymorphisms, subject matters described herein based on the complementary relationship between these sequences are applied to the respective sequences and also to the sequences complementary thereto unless otherwise specified. When the subject matters of the present disclosure are applied to the sequence complementary to each sequence, the sequence recognized by the complementary sequence is read as the sequence complementary to the corresponding sequence described in the present specification throughout the specification according to the common technical knowledge.

### EXAMPLES

### Example 1

### Use of a Single Probe for Detection of Template Oligonucleotide for PON1

Based on the nucleotide sequence comprising the site of the PON1 gene polymorphism (Q192R) rs662 (SEQ ID NO:1), the probe shown in Table 4 having C at its 3′-end was designed. In Table 4, the position of the probe is represented by nucleotide positions in the nucleotide sequence shown in SEQ ID NO:1. Labeling with Pacific Blue was carried out according to a conventional method.

The sequences of the template oligonucleotides (wild-type (SEQ ID NO:18) and mutant type (SEQ ID NO:19)) used as the subject sequences to be detected are shown in Table 4. In Table 4, the positions of the oligonucleotides are represented by nucleotide positions in the nucleotide sequence shown in SEQ ID NO:1. In case of heterozygous type, the template oligonucleotides of SEQ ID NOs:18 and 19 were mixed at a ratio of 1:1 to provide the sample.

TABLE 4

| | | | SEQ ID | | GC content | | Tm | |
|---|---|---|---|---|---|---|---|---|
| Name | Sequence (5′→3′) | Position | NO: | mer | (%) | Tm (mt) | (WT) | ΔTm |
| PON1 (Q192R) probe | tgacccctacttacGatc-(Pacific Blue) | 287-304 | 2 | 18 | 50 | 48.6 | 41 | 7.6 |

*The nucleotide denoted by the upper case letter indicates the position of the mutation.

| Template | | | SEQ ID | |
|---|---|---|---|---|
| Oligonucleotide | Sequence (5′→3′) | Position | NO: | mer |
| Wild-type | cccaggatTgtaagtaggggtcaag | 309-285 | 18 | 25 |
| Mutant-type | cccaggatCgtaagtaggggtcaag | 309-285 | 19 | 25 |

*The nucleotides denoted by the upper case letters indicate the position of the mutation.

Using the reagents described below and a fully automatic SNPs testing device (I-DENSY, manufactured by ARKRAY, Inc.), Tm analysis was carried out to evaluate the performance of the fluorescently labeled oligonucleotide (SEQ ID NO:2).

In the Tm analysis, treatment was carried out at 95° C. for 1 second and then at 40° C. for 60 seconds, followed by increasing the temperature from 40° C. to 75° C. at a heating rate of 1° C./3 seconds, while measuring changes in the fluorescence intensity with time. The excitation wavelength and detection wavelength in the Tm analysis were 365 to 415 nm and 445 to 480 nm, respectively (Pacific Blue).

TABLE 5
(Reaction solution volume: 50 μl)

| | |
|---|---|
| 1 × Gene Taq Universal buffer | |
| (manufactured by Nippon Gene Co., Ltd.) | |
| PON1 (Q192R) probe | 0.2 μM |
| Template oligonucleotide | 0.2 μM |

As a result of the Tm analysis using the probe shown in Table 4, the wild-type oligonucleotide (squares) showed a peak of Pacific Blue at about 55° C., and the mutant-type oligonucleotide (diamonds) showed a peak of Pacific Blue at about 62° C. (FIG. 2). The heterozygous oligonucleotide (triangles) wherein the wild-type and the mutant type are mixed showed peaks of Pacific Blue at about 55° C. and about 61° C. (FIG. 2).

As described above, it was revealed that the wild-type (A/A), heterozygous type (A/G) and mutant type (G/G) may be distinguished from one another since these clearly showed unique patterns of peaks.

### Example 2

### Use of a Single Probe for Detection of Template Oligonucleotide for PON1

Based on the nucleotide sequence comprising the site of the PON1 gene polymorphism (Q192R) rs662 (SEQ ID NO:1), the probe shown in Table 6 having C at its 5′-end was designed. In Table 6, the position of the probe is represented by nucleotide positions in the nucleotide sequence shown in SEQ ID NO:1. Labeling with TAMRA was carried out according to a conventional method.

The sequences of the template oligonucleotides (wild-type (SEQ ID NO:18) and mutant type (SEQ ID NO:19)) in Table 4 were used as the subject sequences to be detected. In case of heterozygous type, the template oligonucleotides of SEQ ID NOs:18 and 19 were mixed at a ratio of 1:1 to provide the sample.

TABLE 6

| | | | SEQ ID | | GC content | | Tm | |
|---|---|---|---|---|---|---|---|---|
| Name | Sequence (5′→3′) | Position | NO: | mer | (%) | Tm (mt) | (WT) | ΔTm |
| PON1 (Q192R) probe2 | (TAMRA)-cccctacttacAatcctg-P | 290-307 | 10 | 18 | 50 | 36.2 | 47.2 | 11 |

*The nucleotide denoted by the upper case letter indicates the position of the mutation. P at the 3′-end represents phosphorylation.

Using the reagents described below and a fully automatic SNPs testing device (I-DENSY, manufactured by ARKRAY, Inc.), Tm analysis was carried out to evaluate the performance of the fluorescently labeled oligonucleotide (SEQ ID NO:10).

In the Tm analysis, treatment was carried out at 95° C. for 1 second and then at 40° C. for 60 seconds, followed by increasing the temperature from 40° C. to 75° C. at a heating rate of 1° C./3 seconds, while measuring changes in the fluorescence intensity with time. The excitation wavelength and detection wavelength in the Tm analysis were 520 to 555 nm and 585 to 700 nm, respectively (TAMRA).

TABLE 7
(Reaction solution volume: 50 μl)

| | |
|---|---|
| 1 × Gene Taq Universal buffer | |
| (manufactured by Nippon Gene Co., Ltd.) | |
| PON1 (Q192R) probe 2 | 0.2 μM |
| Template oligonucleotide | 0.2 μM |

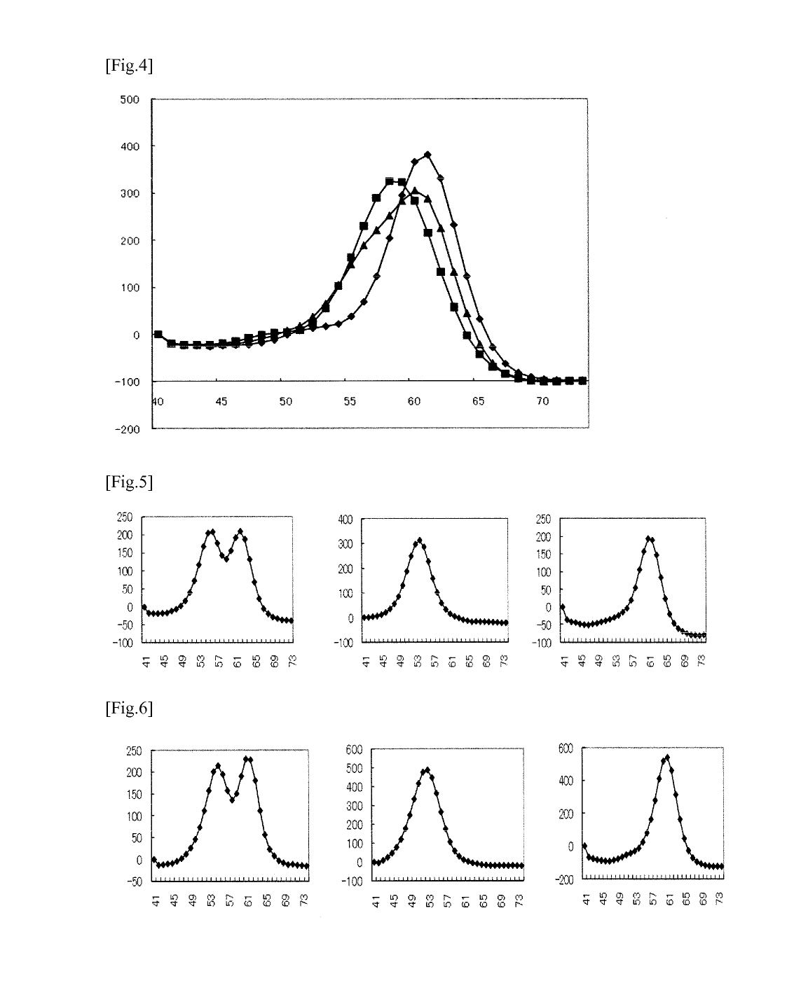

As a result of the Tm analysis using the probe shown in Table 6, the wild-type oligonucleotide (squares) showed a peak of TAMRA at about 60° C., and the mutant-type oligonucleotide (diamonds) showed a peak of TAMRA at about 52° C. (FIG. 4). Therefore, it was revealed that the wild-type and the mutant type may be distinguished from each other.

The heterozygous oligonucleotide (triangles) wherein the wild-type and the mutant type are mixed showed peaks of TAMRA at about 51° C. and about 60° C. (FIG. 4). Therefore, it was revealed that the heterozygous type may be distinguished from the wild-type and the mutant type.

### Comparative Example 1

### Use of a Single Probe for Detection of Template Oligonucleotide for PON1

Based on the nucleotide sequence comprising the site of the PON1 gene polymorphism (Q192R) rs662 (SEQ ID NO:1), the probe shown in Table 8 having C at its 5′-end was designed. In Table 8, the position of the probe is represented by nucleotide positions in the nucleotide sequence shown in SEQ ID NO:1. Labeling with TAMRA was carried out according to a conventional method.

The sequences of the template oligonucleotides in Table 4 (wild-type (SEQ ID NO:18) and mutant type (SEQ ID NO:19)) were used as the subject sequences to be detected. In case of the heterozygous type, the template oligonucleotides of SEQ ID NOs:18 and 19 were mixed at a ratio of 1:1 to provide the sample.

TABLE 8

| | | | SEQ ID | | GC content | Tm | Tm | |
|---|---|---|---|---|---|---|---|---|
| Name | Sequence (5′→3′) | Position | NO: | mer | (%) | (mt) | (WT) | ΔTm |
| PON1 (Q192R) probe 3 | (TAMRA)-cttgacccctacttacGa | 285-302 | 20 | 18 | 50 | 48.9 | 41.2 | 7.6 |

*The nucleotide denoted by the upper case letter indicates the position of the mutation.

Using the reagents described below and a fully automatic SNPs testing device (I-DENSY, manufactured by ARKRAY, Inc.), Tm analysis was carried out to evaluate the performance of the fluorescently labeled oligonucleotide (SEQ ID NO:20).

In the Tm analysis, treatment was carried out at 95° C. for 1 second and then at 40° C. for 60 seconds, followed by increasing the temperature from 40° C. to 75° C. at a heating rate of 1° C./3 seconds, while measuring changes in the fluorescence intensity with time. The excitation wavelength and detection wavelength in the Tm analysis were 520 to 555 nm and 585 to 700 nm, respectively (TAMRA).

TABLE 9
(Reaction solution volume: 50 μl)

| | |
|---|---|
| 1 × Gene Taq Universal buffer | |
| (manufactured by Nippon Gene Co., Ltd.) | |
| PON1 (Q192R) probe 3 | 0.2 μM |
| Template oligonucleotide | 0.2 μM |

As a result of the Tm analysis using the probe shown in Table 8, the wild-type oligonucleotide (squares) showed a peak of TAMRA at about 60° C., and the mutant-type oligonucleotide (diamonds) showed a peak of TAMRA at about 61° C. (FIG. 4). Therefore, it was revealed that these cannot be distinguished from each other because of small A of the Tm value.

Further, the heterozygous oligonucleotide (triangles) wherein the wild-type and the mutant type are mixed showed a peak of TAMRA only at about 60° C. (FIG. 4). Therefore, it was revealed that the heterozygous type shows only a single detection peak and cannot be distinguished from the wild-type and the mutant type.

Accordingly, taking the above results into account together with the results in Examples 1 and 2, it may be understood that the sequence of the probe cannot be arbitrary even in cases where C at the 5′-end or 3′-end of the probe is fluorescently labeled, and that fluorescent labeling of C at position 290 or 304 in the nucleotide sequence shown in SEQ ID NO:1, as is the cases of the probes shown in SEQ ID NOs:2 and 10, is important.

### Example 3

### Use of a Plurality of Probes for Detection from Purified Human Genomic DNA or Whole Blood

Polymorphic regions were amplified as described below from purified human genomic DNA or whole blood by PCR using the primers described below, and Tm analysis was carried out using the probes shown in SEQ ID NOs:2, 24 and 28.

First, based on the nucleotide sequence comprising the site of the PON1 gene polymorphism (Q192R) rs662 (SEQ ID NO:1), the primers shown in Table 10 were designed such that the polymorphic site may be amplified. In Table 10, the positions of the primers are represented by nucleotide positions in the nucleotide sequence shown in SEQ ID NO:1.

Subsequently, based on the nucleotide sequence comprising the site of the CYP2C19*2 gene polymorphism (G681A) (SEQ ID NO:21), primers shown in Table 11 were designed such that the polymorphic site may be amplified. In Table 11, the positions of the primers are represented by nucleotide positions in the nucleotide sequence shown in SEQ ID NO:21. Position 200 in SEQ ID NO:21 is represented by r, which means that the wild-type nucleotide is G and the mutant-type nucleotide is A.

Further, based on the nucleotide sequence comprising the site of the CYP2C19*3 gene polymorphism (G636A) (SEQ ID NO:25), primers shown in Table 11 were designed such that the polymorphic site may be amplified. In Table 11, the positions of the primers are represented by nucleotide positions in the nucleotide sequence shown in SEQ ID NO:25. Position 302 in SEQ ID NO:25 is represented by r, which means that the wild-type nucleotide is G and the mutant-type nucleotide is A.

Thereafter, PCR and Tm analysis were carried out using a fully automatic SNPs testing device (trade name: i-densy IS-5310, manufactured by ARKRAY, Inc.). The composition of the PCR reaction solution was as described below. As a sample, whole blood or purified human genomic DNA as described below was used. The conditions for PCR and Tm analysis were: 95° C. for 60 seconds→(95° C. for 1 second→66° C. for 30 seconds)×50 cycles→95° C. for 1 second→40° C. for 60 seconds→(40° C.→75° C., 1° C./3 seconds).

The excitation wavelength and detection wavelength in the Tm analysis were 365 to 415 nm and 445 to 480 nm, respectively (Pacific Blue); 420 to 485 nm and 520 to 555 nm, respectively (BODIPY FL); or 520 to 555 nm and 585 to 700 nm, respectively (TAMRA).

TABLE 10

| | | | SEQ ID | | | | | |
|---|---|---|---|---|---|---|---|---|
| Name | Sequence (5′→3′) | Position | NO: | mer | | | | |
| PON1 (Q192R) F | gttgctgtgggacctgagcac | 237-257 | 16 | 21 | | | | |
| PON1 (Q192R) R | tctgccaccactcgaacttcacttg | 379-355 | 17 | 25 | | | | |
| | | | SEQ ID | | GC content | Tm | Tm | |
| Name | Sequence (5′→3′) | Position | NO: | mer | (%) | (mt) | (WT) | ΔTm |
| PON1 (Q192R) probe | tgacccctacttacGatc-(Pacific Blue) | 287-304 | 2 | 18 | 50 | 48.6 | 41 | 7.6 |

*The nucleotide denoted by the upper case letter indicates the position of the mutation.

TABLE 11

| | | | SEQ ID | |
|---|---|---|---|---|
| Name | Sequence (5′→3′) | Position | NO: | mer |
| CYP2C19(*2) F | taaattattgtatctatagatatgcaataattttccca | 141-180 | 22 | 40 |
| CYP2C19(*2) R | cccgagggttgttgatgtccatc | 302-280 | 23 | 23 |
| CYP2C19(*2) probe | tcccAggaacccataac-(BODIPY FL) | 196-212 | 24 | 17 |

*The nucleotide denoted by the upper case letter indicates the position of the mutation.

| | | | SEQ ID | |
|---|---|---|---|---|
| Name | Sequence (5′→3′) | Position | NO: | mer |
| CYP2C19(*3) F | tgatggaaaaattgaatgaaaacatcaggattgta | 256-290 | 26 | 35 |
| CYP2C19(*3) R | tcaaaaatgtacttcagggcttggtcaata | 407-378 | 27 | 30 |
| CYP2C19(*3) probe | (TAMRA)-ccctgAatccaggtaag-P | 297-313 | 28 | 17 |

*The nucleotide denoted by the upper case letter indicates the position of the mutation.
P at the 3′-end represents phosphorylation.

TABLE 12 [symbol]
In cases where purified human genomic DNA is used as
the template

[symbol]

| (Reaction solution volume: 50 μl) | |
|---|---|
| 1 × PCR buffer | |
| dNTP | 0.2 mM |
| MgCl₂ | 4.5 mM |
| Taq polymerase (manufactured by | 1.88 U/test |
| ARKRAY, Inc.) | |
| PON1 (Q192R) F | 0.3 μM |
| PON1 (Q192R) R | 1.5 μM |
| CYP2C19(*2) F | 0.3 μM |
| CYP2C19(*2) R | 1.5 μM |
| CYP2C19(*3) F | 0.5 μM |
| CYP2C19(*3) R | 2.5 μM |
| PON1 (Q192R) probe | 0.2 μM |
| CYP2C19(*2) probe | 0.2 μM |
| CYP2C19(*3) probe | 0.2 μM |
| Purified human genomic DNA* | 100 copies |

*Human genomic DNA purified from whole blood is used.

TABLE 13 [symbol]
In cases where whole blood is used as the
template

[symbol]

| (Reaction solution volume: 50 μl) | |
|---|---|
| 1 × PCR buffer | |
| dNTP | 0.2 mM |
| MgCl₂ | 4.5 mM |
| Taq polymerase (manufactured by | 1.88 U/test |
| ARKRAY, Inc.) | |
| PON1 (Q192R) F | 0.3 μM |
| PON1 (Q192R) R | 1.5 μM |
| CYP2C19(*2) F | 0.3 μM |
| CYP2C19(*2) R | 1.5 μM |
| CYP2C19(*3) F | 0.5 μM |
| CYP2C19(*3) R | 2.5 μM |
| PON1 (Q192R) probe | 0.2 μM |
| CYP2C19(*2) probe | 0.2 μM |
| CYP2C19(*3) probe | 0.2 μM |
| Pretreated whole blood | (4 μl*) |

*Pretreated whole blood is used.

### <Purified DNA>

Purified DNA was added to the PCR reaction solution such that 100 copies/test of the DNA was contained therein, and used as a template.

### <Preparation of Whole Blood>

To 70 μl of dilution buffer (1), 10 μl of whole blood was added, and the resulting mixture was mixed well, followed by adding 10 μl of the resulting mixture to 70 μl of dilution buffer (2). By heating 17 μl of this mixture at 95° C. for 10 minutes, 4 μl of pretreated whole blood was obtained. The pretreated whole blood was added to the PCR reaction solution, to use DNA originating from the pretreated whole blood as a template.

TABLE 14

| | |
|---|---|
| Dilution buffer (1) | |
| Tris-HCl (pH 8.0) | 10 mM |
| EDTA (pH 8.0) | 0.1 mM |
| SDS | 0.30% |
| Dilution buffer (2) | |
| Tris-HCl (pH 8.0) | 10 mM |
| 500 mM EDTA (pH 8.0) | 0.1 mM |

As a result of the evaluation of the PON1 gene with the fluorescence of Pacific Blue, the peaks for both the wild-type and the mutant type were found for the purified human genomic DNA (FIG. 5, left panel) and the blood (FIG. 6, left panel). Thus, the samples were revealed to have the heterozygous genotype (A/G).

As a result of the evaluation of the CYP2C19*2 gene with the fluorescence of BODIPY FL, only the peak for the wild-type was found for the purified human genomic DNA (FIG. 5, middle panel) and the blood (FIG. 6, middle panel). Thus, the samples were revealed to have the wild-type genotype (G/G).

As a result of evaluation of the CYP2C19*3 gene with fluorescence of TAMRA, only the peak for the mutant type was found for the purified human genomic DNA (FIG. 5, right panel) and the blood (FIG. 6, right panel). Thus, the samples were revealed to have the mutant-type genotype (A/A).

As can be seen from the results shown in FIGS. 5 and 6, when the probes shown in SEQ ID NOs:2, 24 and 28 were used, changes in the fluorescence intensity that may be analyzed by Tm analysis were observed for the PON1 gene polymorphism and the CYP2C19*2 and CYP2C19*3 gene polymorphisms.

That is, in terms of the PON1 gene polymorphism, the purified human genomic DNA and the whole blood having the heterozygous genotype A/G showed two peaks (at about 56° C. and about 62° C.) and hence exhibited a unique pattern in respect of the amount of change in the fluorescence intensity. Further, also in terms of the CYP2C19*2 gene polymorphism, the purified human genomic DNA and the whole blood showed a single peak (about 54° C., wild-type G/G) and hence exhibited a unique pattern in respect of the amount of change in the fluorescence intensity. Further, also in terms of the CYP2C19*3 gene polymorphism, the purified human genomic DNA and the whole blood showed a single peak (about 62° C., mutant type A/A) and hence exhibited a unique pattern of the amount of change in the fluorescence intensity.

Thus, using the probes of SEQ ID NOs:2, 24 and 28 simultaneously, the PON1 gene polymorphism and the CYP2C19*2 and CYP2C19*3 gene polymorphisms may be detected at once.

Further, since the probe of SEQ ID NO:10 is labeled at C at its end as in the case of the probe of SEQ ID NO:2 and its effect has been demonstrated in Example 2, it is also possible to detect the PON1 gene polymorphism and the CYP2C19*2 and CYP2C19*3 gene polymorphisms at once by simultaneous use of the probe of SEQ ID NO:10 and the probes of SEQ ID NOs:24 and 28.

1. A probe comprising a labeled oligonucleotide selected from P1, P1′, P2 and P2′ below:
(P1) an oligonucleotide of 6 to 53 nucleotides comprising a sequence at least 85% identical to nucleotides 299 to 304 in SEQ ID NO: 1, wherein the nucleotide corresponding to the nucleotide at position 304 in SEQ ID NO: 1 is a cytosine and is labeled;
(P1′) an oligonucleotide of 6 to 53 nucleotides which hybridizes with the complementary nucleotide sequence to the nucleotides 299 to 304 in SEQ ID NO: 1 under stringent conditions, wherein the nucleotide complementary to the nucleotide at position 304 in SEQ ID NO: 1 is a cytosine and is labeled;
(P2) an oligonucleotide of 12 to 54 nucleotides comprising a sequence at least 85% identical to nucleotides 290 to 301 in SEQ ID NO: 1, wherein the nucleotide corresponding to the nucleotide at position 290 in SEQ ID NO: 1 is a cytosine and is labeled; and
(P2′) an oligonucleotide of 12 to 54 nucleotides which hybridizes with the complementary nucleotide sequence to the nucleotides 290 to 301 in SEQ ID NO: 1 under stringent conditions, wherein the nucleotide complementary to the nucleotide at position 290 is a cytosine and is labeled.

2. The probe according to claim 1, wherein the nucleotide corresponding to the nucleotide at position 304 in SEQ ID NO: 1 is labeled with a fluorescent dye, the nucleotide complementary to the nucleotide at position 304 in SEQ ID NO: 1 is labeled with a fluorescent dye, the nucleotide corresponding to the nucleotide at position 290 in SEQ ID NO: 1 is labeled with a fluorescent dye, and the nucleotide complementary to the nucleotide at position 290 is labeled with a fluorescent dye.

3. The probe according to claim 1, wherein
said oligonucleotides P1 and P1′ have the nucleotide corresponding to the nucleotide at position 304 labeled with a fluorescent dye at the first, second or third position counted from the 3′-end; and
said oligonucleotides P2 and P2′ have the nucleotide corresponding to the nucleotide at position 290 labeled with a fluorescent dye at the first, second or third position counted from the 5′-end.

4. The probe according to claim 1, wherein
said oligonucleotides P1 and P1′ have the nucleotide corresponding to the nucleotide at position 304 labeled with a fluorescent dye at the 3′-end; and
said oligonucleotides P2 and P2′ have the nucleotide corresponding to the nucleotide at position 290 labeled with a fluorescent dye at the 5′-end.

5. The probe according to claim 2, wherein said fluorescently labeled oligonucleotide emits fluorescence when the fluorescently labeled oligonucleotide is not hybridized with a target sequence, and the fluorescence intensity decreases or increases when the fluorescently labeled oligonucleotide is hybridized with a target sequence.

6. The probe according to claim 2, wherein said fluorescently labeled oligonucleotide emits fluorescence when the fluorescently labeled oligonucleotide is not hybridized with a target sequence, and the fluorescence intensity decreases when the fluorescently labeled oligonucleotide is hybridized with a target sequence.

7. The probe according to claim 1, wherein
said oligonucleotides P1 and P1′ have 6 to 28 consecutive nucleotides; and
said oligonucleotides P2 and P2′ have 12 to 28 consecutive nucleotides.

8. The probe according to claim 1, wherein
said oligonucleotides P1 and P1′ have 6 to 18 consecutive nucleotides; and
said oligonucleotides P2 and P2′ have 12 to 18 consecutive nucleotides.

9. A method of detecting a polymorphism in a paraoxonase 1 (PON1) gene in a sample containing nucleic acid which may contain said polymorphism comprising contacting the probe according to claim 1 with said sample and detecting the presence or absence of said polymorphism.

10. A method for detecting a polymorphism in the PON1 gene, comprising the Steps (I) to (IV) below:
(I) adding said probe according to claim 1 to a sample containing DNA and allowing said probe to hybridize with said DNA;
(II) changing the temperature to dissociate the hybridization complex between said DNA and said probe and measuring the fluctuation of the signal due to said dissociation of said hybridization complex;
(III) analyzing said fluctuation of the signal to determine the Tm value; and
(IV) determining based on said Tm value the presence of the polymorphism of interest.

11. The method according to claim 10, further comprising amplifying nucleic acid before said Step (I) or at the same time as said Step (I).

12. The method according to claim 9, further comprising detecting in the same system at least one polymorphism selected from a polymorphism of the nucleotide corresponding to the nucleotide at position 200 in SEQ ID NO:21 and a polymorphism of the nucleotide corresponding to the nucleotide at position 302 in SEQ ID NO:25.

13. The method according to claim 9, further comprising detecting in the same system a polymorphism of the nucleotide corresponding to the nucleotide at position 301 in SEQ ID NO:1, a polymorphism of the nucleotide corresponding to the nucleotide at position 200 in SEQ ID NO:21 and a polymorphism of the nucleotide corresponding to the nucleotide at position 302 in SEQ ID NO:25.

14. A method for judging a pharmacological effect of a drug in a subject, comprising detecting a polymorphism in the PON1 gene by the method for detecting a polymorphism according to claim 9 and judging resistance to said drug and/or a pharmacological effect of said drug based on the presence or absence of said polymorphism in a sample from said subject.

15. A reagent kit comprising the probe according to claim 1.

16. The kit according to claim 15, further comprising a primer for amplifying a nucleotide sequence comprising the region with which said probe hybridizes.

17. The kit according to claim 15, further comprising:
a polymorphism detection probe for detecting a polymorphism of the nucleotide corresponding to the nucleotide at position 200 in SEQ ID NO:21; and
a polymorphism detection probe for detecting a polymorphism of the nucleotide corresponding to the nucleotide at position 302 in SEQ ID NO:25.
